SEQUENCE LISTING (1 sequence)

SEQ ID NO: 1 - protein, 175 aa, Homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

(19) United States
(12) Patent Application Publication
(10) Pub. No.: US 2020/0191801 A1
(43) Pub. Date: Jun. 18, 2020

(54) ALPHA-B CRYSTALLIN IN THE DIAGNOSIS OF NEONATAL BRAIN DAMAGE

(71) Applicant: Susanne PANGRATZ-FÜHRER, Stockdorf (DE)

(72) Inventor: Susanne PANGRATZ-FÜHRER, Stockdorf (DE)

(21) Appl. No.: 16/472,816

(22) PCT Filed: Dec. 22, 2017
(86) PCT No.: PCT/EP2017/084354

§ 371 Date: Jun. 21, 2019

(30) Foreign Application Priority Data
Dec. 23, 2016 (EP) 16206577.5

(51) Int. Cl. G01N 33/68

(52) U.S. Cl. CPC G01N 33/6893; G01N 2800/28; G01N 2800/2871

(57) ABSTRACT

The invention provides a method for the diagnosis of brain damage in a neonate comprising a step of analyzing the level of alpha-B crystallin in a tissue or body fluid sample obtained from the neonate. The method is particularly useful for diagnosing preterm infants or neonates with a low birth weight.

### BACKGROUND OF THE INVENTION

Neonatal health care faces many challenges. Newborn children require special medical care and protection, in particular when birth complications occur due to premature delivery. Preterm newborns are susceptible to several high-risk factors, some of which are associated with influencing brain development.

Preterm-birth or premature birth is the birth of a child at a gestational age of less than 37 weeks. Premature infants are at a higher risk for severe diseases, as well as delays in development or hearing and sight problems.

According to the WHO, the number of pre-term births is rising each year, with presently about 1 in 10 infants being born preterm. For instance, in Germany, each year an estimated 63.000 infants are born preterm, with 8.000 infants born before 30 weeks of gestation. The cause of preterm labor still is elusive. Studies have identified several risk factors, but no clear cause.

Pre-term birth complications are the most common cause of death among children under 5 years worldwide. Due to improvements in healthcare, in particular neonatal intensive care, the survival rate of preterm infants has drastically increased in the recent years, thus currently about 90% of preterm infants survive. Unfortunately, the chances of survival without long term difficulties and disabilities are lower.

Many preterm infants suffer brain injuries as a result of the preterm birth. Such injuries to the developing brain can lead to devastating neurologic consequences. The severity of these injuries is inversely related to gestational age and birth weight, with preterm infants being at a much higher risk for long-term neurologic deficits than infants born at a normal gestation age.

Approximately, 50% of very low birth weight newborns (below 1500 g) suffer from a hypoxic-ischemic injury. Periventricular Leukomalacia (PVL) and Intraventricular Hemorrhage (IVH) are the most frequent types of brain injuries of premature infants.

PVL is the leading known cause of cerebral palsy and cognitive deficits, and has also been associated with visual dysfunction and epilepsy (Serdaroglu G, Tekgul H, Kitis O, Serdaroglu E, Gokben S.: Correlative value of magnetic resonance imaging for neurodevelopmental outcome in periventricular leukomalacia. Dev Med Child Neurol. 2004 November; 46(11):733-9; Resić B, Tomasović M, Kuzmanić-Samija R, Lozić M, Resić J, Solak M.: Neurodevelopmental outcome in children with periventricular leukomalacia. Coll Antropol. 2008 January; 32 Suppl 1:143-7; Deng W, Pleasure J, Pleasure D.: Progress in periventricular leukomalacia. Arch Neurol. 2008 October; 65(10):1291-5. doi: 10.1001/archneur.65.10.1291.). IVH can cause injury to the germinal matrix and the subventricular zone. PVL can occur alone or in addition to IVH. There is presently no treatment for PVL or IVH.

The early detection of PVL and IVH is a yet unsolved problem, since only severe injuries can be identified with head ultrasound, the currently most common diagnostic method. Although Diffusion-weighted magnetic resonance imaging (DWI) is more efficient at identifying PVL, it is rarely used for preterm infants to receive an MRI unless they have had a particularly difficult course of development.

Thus, with all efforts focused on survival of preterm infants, PVL and IVH might go unnoticed and a potential time window to interfere with the cascade of damage will pass by. Therefore, there is a need for biomarkers to be able to quickly discriminate infants at risk for injury.

So far, there are only a few biomarkers being studied in preterm and term infants with PVL and IHV, despite the urgent need for biomarkers to screen infants for brain injury and to monitor the progression of disease. Some of the most promising biomarkers for IVH identified so far are S100β and activin. They could potentially be useful in the early detection of brain damage, but unfortunately the level of these biomarkers is also influenced by other factors such as gestational age and intrauterine growth restriction, which unfortunately results in unreliable diagnostic results.

Additionally, reports on biomarkers for PVL are rare. Immuno markers of early stage PVL were discovered through autopsy studies on preterm infants: Human beta-amyloid precursor protein (β-APP) might be a marker of diffuse axonal damage (Arai Y, Deguchi K, Mizuguchi M, Takashima S.: Expression of beta-amyloid precursor protein in axons of periventricular leukomalacia brains. Pediatr Neurol. 1995 September; 13(2):161-3), and fractin could be an apoptopic marker.

### SUMMARY OF THE INVENTION

The present invention relates to a method for the diagnosis of brain damage in a neonate, in particular in a preterm infant. The method involves the analysis of a sample of the newborn for the level of αB-crystallin (also referred to as alpha-B crystallin, CryAB), which can serve as biomarker for the risk and severity of brain damage in newborn infants.

In a yet further aspect, the invention relates to an antibody specific for αB-crystallin for use in a method for the diagnosis and/or prognosis of brain damage in a newborn infant.

### FIGURE LEGEND

FIG. 1: Comparison of the number of infants without elevated αB-crystallin levels (CryAB) (white) and infants with elevated αB-crystallin levels (black) among term-born infants (left) and pre-term born infants (right).

FIG. 2: Illustration of the number of infants with an ultrasound diagnosed brain injury (hatched area) among all infants increased αB-crystallin levels (CryAB).

### DETAILED DESCRIPTION OF THE INVENTION

The inventor identified the need for a new biomarker for the prediction and diagnosis of brain damage in neonates, in particular for the diagnosis of hemorrhagic or ischemic brain damage. It was surprisingly found that the level of αB-crystallin can serve as a biomarker for the prognosis and or diagnosis of brain damage, in particular hemorrhagic and ischemic brain damage in newborn infants, in particular preterm infants.

As such, in a first aspect, the invention relates to a method for the diagnosis and/or prognosis of brain damage in a neonate, the method comprising the following steps:

a) analyzing the level of αB-crystallin in a sample from the neonate; and optionally
b) comparing the level of αB-crystallin to a reference value.

In general, αB-crystallin is a structural protein in the lens of the eye. It is also a member of the family of small heat shock proteins.

In adult patients having suffered a stroke, it is currently thought that αB-crystallin, like several other polypeptides, may also plays a role in brain cell protection, which is yet to be confirmed in clinical studies.

However, the occurrence of αB-crystallin in neonates outside the eye lens, and its levels in tissues and body fluids, have not yet been investigated in detail. In particular, it has been entirely unknown to what extent the αB-crystallin levels in neonates respond to events, such as events associated with birth, or with the development of brain functions, or with potential damage to the brain. It was therefore surprising to find that relevant αB-crystallin levels may be found in neonates, even in non-ophthalmic tissues, and that these levels appear to respond to, or correlate with, damage to the brain as e.g. frequently associated with preterm birth.

One of the major benefits of the diagnostic method according to the invention is that it provides a basis for deciding on further diagnostic and/or therapeutic interventions to be carried out on the neonate. Further diagnosis may include, for example, diffusion tensor imaging (DTI), a technique that allows scanning for microstructural problems in two critical areas of white matter, which are significantly correlated to problems with the child's cognitive and motor development. Therapeutic interventions and Neonatal Intensive Care Unit (NICU) considerations that could potentially be useful in case of elevated levels of αB-crystallin include, without limitation, midline head positioning, delay of procedures requiring excessive handling (such as lumbar puncture), avoidance of sodium bicarbonate infusions and near-infrared spectroscopy monitoring of cerebral oxygenation.

As used herein, the expressions “neonate” and “newborn infant” (or “newborn baby”) are used interchangeably.

Human αB-crystallin has the following protein
sequence (Seq ID No. 1):
MDIAIHHPWI RRPFFPFHSP SRLFDQFFGE HLLESDLFPT
STSLSPFYLR PPSFLRAPSW FDTGLSEMRL EKDRFSVNLD
VKHFSPEELK VKVLGDVIEV HGKHEERQDE HGFISREFHR
KYRIPADVDP LTITSSLSSD GVLTVNGPRK QVSGPERTIP
ITREEKPAVT AAPKK

The method identified by the inventor has been found suitable for the prediction of brain damage in newborn infants. In particular, the method is suitable for the diagnosis of brain damage in pre-term newborns, which are at a particular high risk for developing brain damage.

Within the context of the present invention, a preterm newborn is a infant born before completing 37 weeks of gestation. As such, in one embodiment the invention relates to a method for the diagnosis of brain damage in newborn infants, wherein the newborn was born at a gestational age of 37 weeks or less, specifically less than 37 weeks. In a particular embodiment of the invention, the infant was born at a gestational age of 35 weeks or less. In another embodiment, the infant was born at a gestational age of 32 weeks or less.

Another risk group are newborn infants with low or very low birthweight. In the context of the present invention, low birthweight refers to a birthweight of less than 3000 kg, specifically less than 2800 g, more specifically less than 2500 g. Very low birthweight refers to a birthweight of less than 1500 g.

Accordingly, in one aspect, the invention relates to a method for the diagnosis of brain damage in a newborn infant, wherein the infant has a birthweight of less than 3000 g. In a particular embodiment, the infant has a birthweight of less than 2800 g. In a more particular embodiment, the infant has a birthweight of less than 2500 g, more particularly less than 2000 g. In a further embodiment, the infant has a birthweight of less than 1500 g.

A further risk group for which the method is suitable are newborn infants where complications occurred during or before birth. Accordingly, in a further aspect, the invention relates to a method for the diagnosis or prediction of brain damage due to intra- or post partum complications. These include maternal diabetes with vascular disease, decreased placental blood circulation, congenital infection of the fetus, excessive bleeding from the placenta, very low maternal blood pressure, umbilical cord accidents, prolonged stages of labor and abnormal fetal position.

An additional risk group are newborns wherein the mother was suffering from a disease during pregnancy, in particular shortly before and/or even during birth. As such, in one aspect, the invention relates to a method for the diagnosis of brain damage of newborn infants, wherein the mother had a disease during pregnancy. In a particular embodiment of the invention the mothers had an inflammatory disease during pregnancy.

There are different types of brain damage of which a newborn might suffer. So far, the diagnosis and prognosis could only be performed with an ultrasonic examination of the head of the newborn infant, which is only able to detect some specific and severe kinds of brain damage, or with a MRI (DWI) analysis, which is complex and costly.

The inventor surprisingly found that αB-crystallin is a suitable indicator for different types of brain damage. In other words, this biomarker is not limited to a particular brain damage, in contrast to e.g. ultrasonic analysis. In a particular embodiment, the invention therefore relates to a method for the diagnosis of brain damage of newborn infants, wherein the brain damage is diffuse, inflammatory, ischemic or hemorrhagic brain damage.

In one embodiment, the brain damage is ischemic or hemorrhagic brain damage. In a particular embodiment, the method is for diagnosis and prognosis of Periventricular Leukomalacia, Intraventricular Hemorrhage or cerebral palsy.

The sample to be analyzed is any suitable sample obtained from the newborn infant. Preferably the sample is a sample of a bodily fluid. More preferably, the sample is a blood sample, spinal fluid sample, urine sample or sputum sample. More preferably, the sample is a blood sample or derived from a blood sample, such as blood plasma or serum. For example, the blood sample may be umbilical cord blood, or the plasma fraction thereof. Alternatively, the blood sample may have been obtained from any other vascular access.

In order to provide a rapid and reasonably fast analysis, which allows medical and pharmaceutical intervention if necessary, it is preferred that that the sample was taken within the first few hours after birth. Preferably, the sample was taken within the first two hours, more preferably within the first hour after birth, in particular within the first hour after cutting the umbilical cord. As mentioned, if elevated levels of αB-crystallin are found, in particular levels above the reference value as discussed below, this may indicate that further diagnostic procedures and/r therapeutic inventions are indicated, and can be initiated without further delay,

The analysis of αB-crystallin may be performed with any suitable analytical method which allows at least the detection of αB-crystallin. Preferably the method allows qualitative and quantitative analysis of αB-crystallin. Ideally, the method would allow a rapid analysis of αB-crystallin, preferably qualitatively and quantitatively.

Since, the concentration of αB-crystallin in a sample of a healthy newborn is rather low, it is preferred that the analytical method is sufficiently sensitive to allow the determination of levels of αB-crystallin of as low as 0.1 ng/ml, or as low as 0.05 ng/ml, or even lower than 0.05 ng/ml. In a preferred embodiment, the analytical method is an antibody-based method or a mass-spectroscopic method.

In one specific embodiment, the analysis is performed with an antibody based assay, preferably an ELISA assay. Alternatively, the analysis might be performed using a standardized western blot or dot-blot assay.

In an alternative embodiment of the invention, the analysis is performed using a mass-spectrometric method. Most preferably, the mass spectrometric method allows the detection and quantification of αB-crystallin. In one embodiment, the mass spectrometric method is a direct MS method. In a preferred embodiment, the method is coupled with a chromatographic method. In another preferred embodiment, the analysis is performed with a LC/MS, preferably HPLC/MS method. An example of a suitable method for αB-crystallin detection and quantification is provided in Rothbard J B, Zhao X, Sharpe O, Strohman M J, Kurnellas M, Mellins E D, Robinson W H, Steinman L., J Immunol. 2011, Apr. 1; 186(7).

With respect to the mass spectroscopic analysis, in particular LC/MS or HPLC/MS analysis, the invention further relates to a purified αB-crystallin protein for use as a standard in the assessment. Preferably, said purified protein comprises SEQ ID NO. 1 More preferably, said purified protein consists of SEQ ID No. 1.

It was found that a level of αB-crystallin of more than 0.1 ng/mL, preferably more than 0.5 ng/mL, indicates an increased risk of brain damage in newborns. In particular, it was surprisingly found that the level of αB-crystallin is indicative for the risk and severity of potential brain damage of a newborn.

Usually the level of αB-crystallin is at or below the detection limit of an ELISA assay for αB-crystallin. The inventor found that an αB-crystallin level of up to 0.1 ng/ml, preferably more than 0.5 ng/mL, αB-crystallin in sample of a newborn is suitable as a reference value and in most cases not indicative for brain damage. A level higher than 0.1 ng/ml, preferably more than 0.5 ng/mL, indicates a risk for brain damage, with an increased risk associated with increased levels.

In a further aspect, the invention relates to an antibody or antibody fragment for use in a diagnostic or prognostic method to predict brain damage in newborn infants as described above.

Preferably the antibody is suitable for detection of αB-crystallin in an antibody based assay, such as ELISA or dot-blot. The antibody may be a polyclonal or monoclonal antibody. In one embodiment of the invention, the antibody is a polyclonal antibody. In an alternative embodiment the antibody is a monoclonal antibody.

The antibody might be coupled to a detectable compound. In one embodiment of the invention the antibody is coupled to a fluorescent dye. In an alternative embodiment the antibody is coupled to an enzyme capable of generating a detectable signal, such as horseradish peroxidase. In a further alternative embodiment, the detectable compound is an affinity tag, such as biotin.

### EXAMPLES

In this pilot study, the αB-crystallin concentration was analyzed from plasma of 52 premature infants (born at less than 35 weeks' gestation) and compared to samples taken from 40 term infants. Thus, we developed a baseline concentration of αB-crystallin as healthy controls.

Plasma samples of preterm infants were collected on day 1 during the first hour after birth from cord blood, and then repeated on day 3 together with routine blood draws. In healthy term infants, cord blood and a sample at day 3 at the time of standard newborn screening were obtained.

All infants underwent a detailed clinical evaluation including head ultrasound for preterm infants and neonatal fundus examination. The blood samples were stored in EDTA tubes, placed on ice for transport and processed within 1 hour. The tubes were centrifuged at 3500 g for 5 minutes at 4° C. The plasma fraction was separated and aliquoted into separate tubes stored at −80° C. prior to processing. If the volume of the blood sample permitted, it was also screened for inflammatory cytokines i.e. IL-6, IL-15-α and TNF-α, which have been associated with white matter injury and cerebral palsy.

Initially, plasma levels of αB-crystallin were assessed using a αB-crystallin-specific ELISA kit (Stressmarq Inc) according to the manufacturer's protocol. To simplify blood sample collection and processing, the methodology was changed from ELISA to using Dried Blood Spots on Newborn Screening Cards for analysis via liquid chromatography tandem mass spectrometry, a technology that allows rapid determination and quantification of CryAB from a single dried blood spot.

The results of the analyses are shown in tables 1 and 2 below.

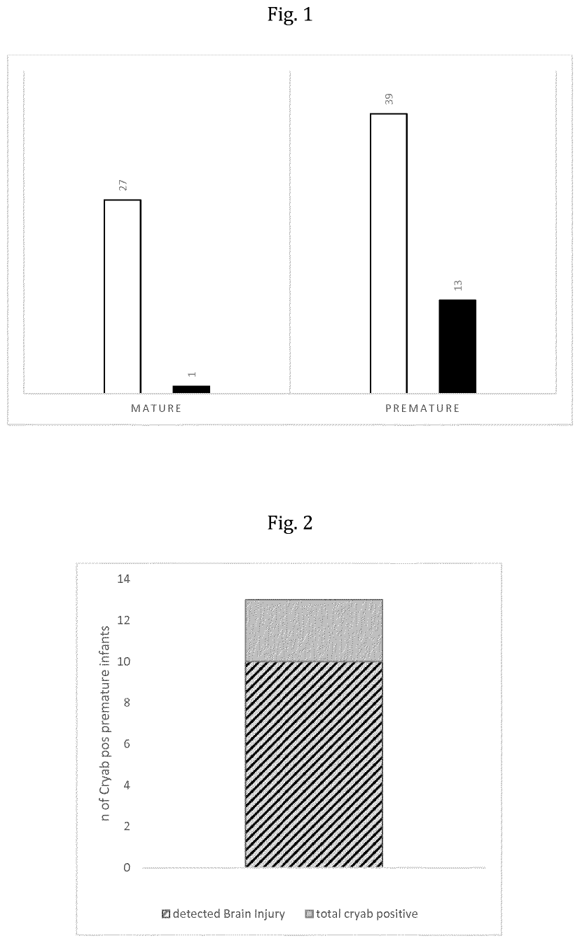

For mature infants, only one out of 27 infants (3.7%) had increased αB-crystallin levels, while for the cohort of premature infants, elevated αB-crystallin levels were detected in 13 out of 52 neonates (25%) (see FIG. 1). Ultrasound examination or neurodevelopmental examination verified that brain injury existed in 10 out of these 13 premature infants (see FIG. 2). Interestingly, for preterm infant no. 10 with the highest αB-crystallin level, ultrasound examination was not able to show brain injury, but neurodevelopmental examination revealed potential white matter damage. At 3-months age, this patient presented with significantly delayed motor development in the leg movements; both the timeline and symptoms being typical markers of the developmental delay associated with (diffuse) Periventricular Leukomalacia (PVL) (cf. Fetters, L; Chen, Yp; Jonsdottir, J; Tronick, Ez (April 2004). “Kicking coordination captures differences between full-term and premature infants with white matter disorder”. Human movement science. 22 (6): 729-48.). It is therefore assumed that all cases of increased αB-crystallin levels without ultrasound findings will subsequently be confirmed to be previously unidentified brain injury.

On the other hand, there were four patients identified by ultrasound with an intracerebral hemorrhage score of 1 to 2 (abbreviated ICH ° I-II) without elevated αB-crystallin levels. For these infants, it is assumed that the damaging incident happened at least 24 hours before birth so that the increased αB-crystallin levels were no longer present at the time of sample withdrawal.

The following tables provide an overview on the determined αB-crystallin levels of the newborns. Missing values indicate that no alpha-B chrystallin levels could be determined.

TABLE 1
αB-crystallin levels determined in term newborn infants

| | αB-crystallin | | | |
|---|---|---|---|---|
| | level ng/ml | αB-crystallin | | Gestational |
| | umbilical cord | level ng/ml | Birthweight | age |
| No. | blood | serum | [g] | [weeks] |
| 1 | 0.10 | 0.10 | 3210 | 39 + 0 |
| 3 | 0.10 | 0.10 | 3220 | 41 + 2 |
| 4 | 0.10 | 0.10 | 3080 | 39 + 3 |
| 5 | 0.10 | 0.10 | 3225 | 38 + 5 |
| 6 | 0.10 | 0.10 | 3600 | 39 + 1 |
| 8 | 0.10 | 0.10 | 3205 | 38 + 2 |
| 9 | 0.10 | 0.10 | 4130 | 41 + 5 |
| 10 | 0.10 | n.a. | 3260 | 39 + 0 |
| 11 | 0.10 | 0.10 | 3200 | 41 + 2 |
| 12 | 0.10 | 0.10 | 5020 | 40 + 3 |
| 16 | 0.41 | n.a. | 2975 | 39 + 3 |
| 19 | n.a. | 0.1 | 4140 | 39 + 1 |
| 21 | 0.13 | n.a. | 2690 | 37 + 5 |
| 22 | 0.10 | 0.10 | 3750 | 41 + 0 |
| 24 | 0.10 | n.a. | 3300 | 41 + 3 |
| 25 | 0.10 | n.a. | 3110 | 39 + 6 |
| 30 | 0.10 | n.a. | 4015 | 40 + 5 |
| 32 | 0.10 | 0.10 | 3655 | 38 + 3 |
| 34 | 0.10 | n.a. | 4255 | 40 + 5 |
| 35 | 0.10 | n.a. | 3405 | 38 + 0 |
| 36 | 0.10 | 0.10 | 3180 | 40 + 1 |
| 39 | 0.16 | n.a. | 3200 | 40 + 2 |
| 40 | 0.10 | n.a. | 3510 | 41 + 3 |
| 41 | 0.07 | n.a. | 1975 | 37 + 6 |
| 42 | 0.10 | n.a. | 2720 | 37 + 6 |
| 43 | 0.10 | n.a. | 3150 | 39 + 2 |
| 44 | 0.10 | n.a. | 2960 | 40 + 2 |

TABLE 2
αB-crystallin levels determined in preterm newborn infants

| | αB-crystallin | | | | |
|---|---|---|---|---|---|
| | level ng/ml | αB-crystallin | Birth | Gestational | Ultrasound/ |
| | umbilical cord | level ng/ml | weight | age | neurodevelopmental |
| No. | blood | serum plasma | [g] | [weeks] | diagnosis |
| 1 | 0.100 | n.a. | 2540 | 35 + 0 | n.a. |
| 2 | 0.100 | 0.1 | 1985 | 35 + 0 | n.a. |
| 3 | 0.155 | 0.1 | 950 | 27 + 2 | ICH °II left |
| 4 | 0.320 | 0.1 | 1750 | 31 + 6 | small plexus cyst |
| 5 | 0.100 | 0.1 | 2290 | 34 + 0 | ICH I° right. |
| 6 | n.a. | 0.1 | 1110 | 35 + 1 | normal |
| 7 | 0.10 | 0.1 | 1860 | 32 + 5 | Ventricular |
| | | | | | asymmetry |
| 8 | 5.708 | 0.1 | 1150 | 27 + 5 | ICH °II |
| 9 | 0.1 | 0.1 | 1235 | 28 + 4 | normal |
| 10 | 19.020 | 0.1 | 1140 | 28 + 4 | Days 1 & 5: |
| | | | | | ultrasound normal, |
| | | | | | but at 3 months |
| | | | | | significantly delayed |
| | | | | | motor development |
| | | | | | (compared to twin |
| | | | | | no. 9). Suspected |
| | | | | | periventricular |
| | | | | | leukomalacia (PVL) |
| 12 | 0.10 | 0.1 | 2169 | 35 + 0 | Normal |
| 13 | 0.10 | n.a. | 840 | 26 + 3 | ventriculomegaly |
| 14 | 0.100 | n.a. | 1890 | 34 + 4 | ICH °I (right), day 1, |
| | | | | | 3 and 7). |
| 15 | 0.10 | n.a. | 1930 | 34 + 4 | ICH °I (l), solitary |
| | | | | | plexus cyst |
| 16 | 0.10 | 0.1 | 2420 | 33 + 6 | Normal |
| 17 | 0.10 | 0.1 | 2200 | 32 + 1 | plexus cyst |
| 18 | 0.10 | 0.1 | 2340 | 33 + 4 | Normal |
| 19 | 2.10 | 0.1 | 1045 | 27 + 4 | ICH°II (r), ICH°III (l) |
| | | | | | posthemorrhagic |
| | | | | | hydrocephalus |
| 20 | 0.10 | n.a. | 880 | 27 + 4 | ICH°II |
| 21 | 0.10 | 0.1 | 1850 | 32 + 6 | Normal |
| 22 | 0.10 | 0.1 | 2090 | 32 + 6 | Normal |
| 23 | 0.30 | 0.1 | 1920 | 34 + 1 | ICH °II |
| 24 | 0.10 | n.a. | 2380 | 34 + 5 | Normal |
| 26 | 0.10 | n.a. | 1655 | 31 + 4 | Normal |
| 27 | 0.10 | n.a. | 1890 | 28 + 3 | Normal |
| 28 | 0.10 | n.a. | 1650 | 34 + 0 | Normal |
| 29 | 0.14 | n.a. | 2010 | 34 + 0 | Normal |
| 31 | 0.10 | 0.1 | 1450 | 29 + 1 | Normal |
| 32 | 0.35 | 0.1 | 960 | 29 + 1 | PVL, ICH I° |
| 37 | 0.10 | 0.1 | 1550 | 32 + 5 | Normal |
| 38 | 0.10 | 0.1 | 1480 | 31 + 2 | Normal |
| 39 | 0.10 | 0.1 | 1800 | 31 + 2 | Normal |
| 40 | 0.83 | 0.1 | 1900 | 31 + 5 | Ventricular |
| | | | | | asymmetry |
| 41 | 0.10 | 0.1 | 2360 | 32 + 5 | Normal |
| 42 | 1.6 | 0.1 | 990 | 29 | ICH °II |
| 43 | 0.1 | 0.1 | 1235 | 28 + 2 | Normal |
| 44 | 0.1 | 0.1 | 1320 | 29 + 6 | Normal |
| 45 | 0.1 | 0.1 | 1690 | 33 + 0 | Ventricular |
| | | | | | asymmetry |
| 46 | 0.1 | n.a. | 1470 | 33 + 0 | Ventricular |
| | | | | | asymmetry |
| 47 | 0.1 | n.a. | 1330 | 33 + 0 | Normal |
| 48 | 0.1 | 0.1 | 1790 | 32 + 5 | Plexus cyst |
| 49 | 0.1 | 0.1 | 1800 | 32 + 5 | normal |
| 50 | 0.45 | 0.1 | 1045 | 28 + 2 | ICH °II, plexus cyst |
| 51 | 0.1 | 0.1 | 1150 | 28 + 2 | Ventricular |
| | | | | | asymmetry |
| 53 | 4.8 | 0.1 | 1470 | 27 + 2 | ICH °II |
| 54 | 0.1 | n.a. | 1415 | 30 + 0 | normal |
| 55 | 2.3 | 0.1 | 740 | 27 + 5 | PVL |
| 56 | 0.1 | 0.1 | 720 | 27 + 5 | Ventricular |
| | | | | | asymmetry |
| 57 | 0.1 | 0.1 | 1450 | 31 + 2 | normal |
| 58 | 0.1 | 0.1 | 1670 | 30 + 2 | normal |
| 59 | 0.1 | n.a. | 2100 | 31 + 2 | Plexus cyst |
| 60 | 0.1 | n.a. | 2050 | 31 + 2 | normal |

What is claimed:

1. A method for the diagnosis of brain damage in a neonate, the method comprising,
a) analyzing the level of alpha-B crystallin in a sample from the neonate; and optionally
b) comparing the level of alpha-B crystallin to a reference value, wherein the sample is a blood sample.

2. The method according to claim 1, wherein the sample is a blood, plasma, serum, sputum, spinal fluid or urine sample.

3. The method according to claim 1, wherein the neonate is a preterm infant born at a gestation age of 37 weeks or less.

4. The method according to claim 1, wherein the neonate has a birthweight of less than 2800 g.

5. The method according to claim 1, wherein the sample was taken from the neonate within 1 h after birth.

6. The method according to claim 1, wherein the diagnosed brain damage is inflammation, ischemic or hemorrhagic brain damage.

7. The method according to claim 6, wherein the diagnosed brain damage is Periventricular Leukomalacia or Intraventricular Hemorrhage.

8. A method according to claim 1, wherein the level of alpha-B crystallin is detected using an antibody against alpha-B crystalline.
